(19) United States
(12) Patent Application Publication
(10) Pub. No.: US 2011/0293576 A1
(43) Pub. Date: Dec. 1, 2011

(54) MESENCHYMAL STROMAL CELL POPULATIONS AND METHODS OF ISOLATING AND USING SAME

(75) Inventors: Claudia Lange, Hamburg (DE); Christopher Westenfelder, Salt Lake City, UT (US)

(73) Assignee: ALLOCURE INC., Burlington, MA (US)

(21) Appl. No.: 13/057,698

(22) PCT Filed: Aug. 4, 2009
(86) PCT No.: PCT/US2009/052733

§ 371 Date: Aug. 8, 2011

Related U.S. Application Data
(60) Provisional application No. 61/086,033, filed on Aug. 4, 2008.

(51) Int. Cl. A61K 35/28; A61P 13/12; A61P 29/00; C12N 5/077; A61P 25/00

(52) U.S. Cl. 424/93.7; 435/325; 435/366

(57) ABSTRACT

The invention relates to mesenchymal stromal cells produced by culturing the cells in platelet lysate supplemented media and methods of using these cells to treat neurological and kidney associated disorders.

### FIELD OF THE INVENTION

The present invention generally relates to mesenchymal stromal cell populations, methods of isolating these populations and methods for treating organ dysfunction, multi-organ failure, cerebral dysfunction and renal dysfunction, including, but not limited to stroke, acute renal failure, transplant associated acute renal failure, graft versus host disease, chronic renal failure, and wound healing.

### BACKGROUND OF THE INVENTION

Stroke or cerebral vascular accident (CVA) is a clinical term for a rapidly developing loss of brain function, due to lack of blood supply. The reason for this disturbed perfusion of the brain can be thrombosis, embolism or hemorrhage. Stroke is a medical emergency and the third leading course of death in Western countries. It is predicted that stroke will be the leading course of death by the middle of this century. This factors for stroke include advanced age, previous stroke or ischemic attack, high blood pressure, diabetes, mellitus high cholesterol, cigarette smoking and cardiac arrhythmia with atrial fibrillation. Therefore, a great need exists to provide a treatment for stroke.

Multi-organ failure (MOF) also remains a major unresolved medical problem. MOF develops in the most severely ill patients who have sepsis, particularly when the latter develops after major surgery or trauma. It occurs also with grater frequency and severity in elderly patients, those with diabetes mellitus, underlying cardiovascular disease and impaired immune defenses. MOF is characterized by shock, acute renal failure (ARF), leaky cell membranes, dysfunction of lungs, liver, heart, blood vessels and other organs. Mortality due to MOF approaches 100% despite the utilization of the most aggressive forms of therapy, including intubation and ventilatory support, administration of vasopressors and antibiotics, steroids, hemodialysis and parenteral nutrition. Many of these patients have serious impairment of the healing of surgical or trauma wound, and, when infected, these wounds further contribute to recurrent infections, morbidity and death.

ARF is defined as an acute deterioration in renal excretory function within hours or days, resulting in the accumulation of “uremic toxins,” and, importantly, a rise in the blood levels of potassium, hydrogen and other ions, all of which contribute to life threatening multisystem complications such as bleeding, seizures, cardiac arrhythmias or arrest, and possible volume overload with pulmonary congestion and poor oxygen uptake. The most common cause of ARF is an ischemic insult of the kidney resulting in injury of renal tubular and postglomerular vascular endothelial cells. The principal etiologies for this ischemic form of ARF include intravascular volume contraction, resulting from bleeding, thrombotic events, shock, sepsis, major cardiovascular surgery, arterial stenoses, and others. Nephrotoxic forms of ARF can be caused by radiocontrast agents, significant numbers of frequently used medications such as chemotherapeutic drugs, antibiotics and certain immunosuppressants such as cyclosporine. Patients most at risk for all forms of ARF include diabetics, those with underlying kidney, liver, cardiovascular disease, the elderly, recipients of a bone marrow transplant, and those with cancer or other debilitating disorders.

Both ischemic and nephrotoxic forms of ARF result in dysfunction and death of renal tubular and microvascular endothelial cells. Sublethally injured tubular cells dedifferentiate, lose their polarity and express vimentin, a mesenchymal cell marker, and Pax-2, a transcription factor that is normally only expressed in the process of mesenchymal-epithelial transdifferentiation in the embryonic kidney. Injured endothelial cells also exhibit characteristic changes.

The kidney, even after severe acute insults, has the remarkable capacity of self-regeneration and consequent re-establishment of nearly normal function. It is thought that the regeneration of injured nephron segments is the result of migration, proliferation and redifferentation of surviving tubular and endothelial cells. However, the self-regeneration capacity of the surviving tubular and vascular endothelial cells may be exceeded in severe ARF. Patients with isolated ARF from any cause, i.e., ARF that occurs without MOF, continue to have a mortality in excess of 50%. This dismal prognosis has not improved despite intensive care support, hemodialysis, and the recent use of atrial natriuretic peptide, Insulin-like Growth Factor-I (IGF-I), more biocompatible dialysis membranes, continuous hemodialysis, and other interventions. An urgent need exists to enhance the kidney's self-defense and autoregenerative capacity after severe injury.

Another acute form of renal failure, transplant-associated acute renal failure (TA-ARF), also termed early graft dysfunction (EGD), commonly develops upon kidney transplantation, mainly in patients receiving transplants from cadaveric donors, although TA-ARF may also occur in patients receiving a living related donor kidney. Up to 50% of currently performed kidney transplants utilize cadaveric donors. Kidney recipients who develop significant TA-ARF require treatment with hemodialysis until graft function recovers. The risk of TA-ARF is increased with elderly donors and recipients, marginal graft quality, significant comorbidities and prior transplants in the recipient, and an extended period of time between harvest of the donor kidney from a cadaveric donor and its implantation into the recipient, known as “cold ischemia time.” Early graft dysfunction or TA-ARF has serious long-term consequences, including accelerated graft loss due to progressive, irreversible loss in kidney function that is initiated by TA-ARF, and an increased incidence of acute rejection episodes leading to premature loss of the kidney graft. Therefore, a great need exists to provide a treatment for early graft dysfunction due to TA-ARF or EGD.

Chronic renal failure (CRF) or Chronic Kidney Disease (CKD) is the progressive loss of nephrons and consequent loss of renal function, resulting in End Stage Renal Disease (ESRD), at which time patient survival depends on dialysis support or kidney transplantation. The progressive loss of nephrons, i.e., glomeruli, tubuli and microvasculature, appears to result from self-perpetuating fibrotic, inflammatory and sclerosing processes, most prominently manifested in the glomeruli and renal interstitium. The loss of nephrons is most commonly initiated by diabetic nephropathy, glomerulonephritides, many proteinuric disorders, hypertension, vasculitic, inflammatory and other injuries to the kidney. Currently available forms of therapy, such as the administration of angiotensin converting enzyme inhibitors, angiotensin receptor blockers, other anti-hypertensive and anti-inflammatory drugs such as steroids, cyclosporine and others, lipid lowering agents, omega-3 fatty acids, a low protein diet, and optimal weight, blood pressure and blood sugar control, particularly in diabetics, can significantly slow and occasionally arrest the chronic loss of kidney function in the above conditions. The development of ESRD can be prevented in some compliant patients and delayed others. Despite these successes, the annual growth of patient numbers with ESRD, requiring chronic dialysis or transplantation, remains at 6%, representing a continuously growing medical and financial burden. There exists an urgent need for the development of new interventions for the effective treatment of CRF or CKD and thereby ESRD, to treat patients who fail to respond to conventional therapy, i.e., whose renal function continues to deteriorate. Stem cell treatment will be provided to arrest/reverse the fibrotic processes in the kidney.

Taken together, therapies that are currently utilized in the treatment of stroke, ARF, the treatment of established ARF of native kidneys per se or as part of MOF, and ARF of the transplanted kidney, and organ failure in general have not succeeded to significantly improve morbidity and mortality in this large group of patients. Consequently, there exists an urgent need for the improved treatment of MOF, renal dysfunction, and organ failure.

Very promising pre-clinical studies in animals and a few early phase clinical trials administer bone marrow-derived hematopoietic stromal cells for the repair or protection of one specific organ such as the heart, small blood vessels, brain, spinal cord, liver and others. These treatments have generally used only a single population of bone-marrow stem cells, either Hematopoietic (HSC) or Mesenchymal stromal cells (MSC), and obtained results are very encouraging in experimental stroke, spinal cord injury, and myocardial infarction. The intracoronary administration of stem cells in humans with myocardial infarction or coronary artery disease has most recently been reported to result in significant adverse events such as acute myocardial infarction, other complications and death. Peripheral administration of stem cells or the direct injection into the injured myocardium showed more favorable results both in animal and Phase I trials. MSC have been infused into patients either simultaneously or a few weeks after they first received a bone marrow transplant in the treatment of cancers, leukemias, osteogenesis imperfecta, and Hurler's syndrome to accelerate reconstitution of adequate hematopoiesis. Effective treatment of osteogenesis imperfecta and Hurler's syndrome has been shown using MSC. Importantly, administration of a mixture of HSC and MSC, known to physiologically cooperate in the maintenance of hematopoiesis in the bone marrow, has, until now (see below) not been utilized for the treatment of any of the above listed renal disorders, MOF or wound healing.

### SUMMARY OF THE INVENTION

The invention encompasses mesenchymal stromal cells that are isolated from bone marrow and methods of producing these mesenchymal stromal cells. The bone marrow is cultured on tissue culture plates for 2-10 days. After this period, non-adherent cells are removed and the remaining adherent cells are cultured for an additional 9-20 days in platelet lysate (PL)-supplemented media. In some embodiments, when the cells reach 80-90% confluence, the cells are removed from the tissue culture plates. These cells are between 85 and 95% mesenchymal stromal cells (MSCs). The cells are then suspended in physiologically acceptable solution with approximately 5% serum albumin and 10% DMSO and frozen at rate of 1° C. per minute temperature decrease.

The invention also encompasses mesenchymal stromal cells that have been cultured in platelet lysate supplemented culture media and wherein the population of mesenchymal stromal cells expresses Pickle1 at a higher degree than mesenchymal stromal cells that have been cultured in fetal calf serum supplemented culture media. In some embodiments, the mesenchymal stromal cells of the invention are less immunogenic than mesenchymal stromal cells that have been cultured in fetal calf serum supplemented culture media.

The invention also encompasses mesenchymal stromal cells that express the antigens CD105, CD90, CD73 and MHC I on their surfaces. In some embodiments, the mesenchymal stromal cells of the invention do not express a protein selected from the group consisting of CD45, CD34 and CD14 on its surface.

The invention also provides methods of using the MSCs of the invention, cultured in PL-supplemented media. These methods include administering the MSCs of the invention to subjects for the treatment of neurological, inflammatory or renal disorders. These disorders include stroke, acute renal failure, transplant associated acute renal failure, graft versus host disease, chronic renal failure, and wound healing. The MSCs are thawed in a step-wise manner, if frozen and the DMSO is diluted from the MSCs. The MSCs are administered intra-arterially to the supra-renal aorta generally by way of the femoral artery. The catheter used to administer the cells, generally is relatively small to minimize damage to the vasculature of the subject. Also, the MSCs of the invention are administered at 50% higher pressure than that in the aorta. The MSCs are administered at a dose of approximately between 10⁵ and 10¹⁰ cells per kg body weight of the subject. Preferably the MSCs are administered at a dose of approximately between 10⁶ and 10⁸ per kg body weight of the subject. These doses of MSCs are suspended in greater than 40 mL of physiologically acceptable carrier with 5% of serum albumin. The volume and serum albumin prevent the MSCs from clumping when they are administered which can lead to side effects in the subject. The cells are administered through the catheter at a rate of about 1 mL of cells per second. Single or multiple administrations of MSCs are used to have therapeutic effect.

The invention also encompasses methods of isolating a population of mesenchymal stromal cells comprising providing bone marrow; culturing the bone marrow on tissue culture plates in culture media between 2 and 10 days; removing non-adherent cells; culturing the adherent cells between 9 and 20 days in platelet lysate supplemented media; and removing the adherent cells from the tissue culture plates; thereby isolating a population of mesenchymal stromal cells. In certain embodiments, the mesenchymal stromal cells are mammalian. In some embodiments, the mammalian mesenchymal stromal cells are human. In some specific embodiments, the platelet lysate is present in the culture media at about 20 μl of platelet lysate per 1 ml of culture media. In other specific embodiments, the platelet lysate is made up of pooled thrombocyte concentrates or pooled buffy coats after centrifugation.

### BRIEF DESCRIPTION OF THE DRAWINGS

FIG. 1 is a photograph of stained MSCs colony forming unit-fibroblast (CFU-F) in media supplemented with fetal calf serum (FCS) or platelet lysate (PL) and plated at the same density.

FIG. 2 is a graph showing the cumulative cell numbers of MSCs grown in media supplemented with fetal calf serum (FCS) or platelet lysate (PL).

FIG. 3 is a bar graph showing downregulation of genes involved in fatty acid metabolism in MSCs cultured in PL-supplemented media.

FIG. 4 is a bar graph showing the relative percentage of Ki-67+ CD3+ cells in the presence of effector (E), irradiated activator (A), and/or PL-generated MSCs (M) in various ratios.

FIG. 5 is a bar graph showing downregulation of MHC II compounds in MSCs cultured in PL-supplemented media when compared to MSCs cultured in FCS-supplemented media.

FIG. 6 is a bar graph showing downregulation of genes associated with cellular adhesion and cellular matrix in MSCs cultured in PL-supplemented media when compared to MSCs cultured in FCS-supplemented media.

FIG. 7 is a bar graph showing relative survival rates of kidney cells rescued with different media after a chemically simulated ischemia event. MSCs from three different donors were used to generate the conditioned media.

### DETAILED DESCRIPTION OF THE INVENTION

The present invention provides mesenchymal stromal cells (MSCs) with unique properties beneficial for their use to treat neurological or kidney pathology. The present invention also provides methods of producing MSCs with unique properties beneficial for their use to treat stroke and kidney pathology. The present invention also provides methods of using MSCs with unique properties beneficial for their use to treat stroke and kidney pathology.

Mesenchymal Stromal Cells Cultured in Platelet Lysate (PL) Supplemented Media

The invention provides mesenchymal stromal cells (MSCs) with unique properties that make them particularly beneficial for use in the treatment of neurological or kidney pathology. The MSCs of the invention are grown in media containing platelet lysate (PL), as described in greater detail below. The culturing of MSCs in PL-supplemented media creates MSCs that are more protective against ischemia-reperfusion damage than MSCs grown in fetal calf serum (FCS)-supplemented media.

The MSCs of the invention, cultured in PL-supplemented media constitute a population with (i) surface expression of the antigens CD105, CD90, CD73 and MHC I, but lacking hematopoietic markers CD45, CD34 and CD14; (ii) preservation of the multipotent trilineage (osteoblasts, adipocytes and chondrocytes) differentiation capability after expansion with PL, however the adipogenic differentiation was delayed and needed longer times of induction. This decreased adipogenic/lipogenic ability is a favourable property because in mice the intraarterial injection of MSCs for treatment of chronic kidney injury has revealed formation of adipocytes (Kunter U, Rong S, Boor P, et al. Mesenchymal stromal cells prevent progressive experimental renal failure but maldifferentiate into glomerular adipocytes. J Am Soc Nephrol 2007 June; 18(6):1754-64). These results are reflected in the gene expression profile of PL-generated cells revealing a downregulation of genes involved in fatty acid metabolism, described in greater detail below.

The MSCs of the invention, cultured in PL-supplemented media have been described to act immunomodulatory by impairing T-cell activation without inducing anergy. There is a dilution of this effect in vitro in mixed lymphocyte cultures (MLC) leading eventually to an activation of T-cells if decreasing amounts of MSCs, not cultured in PL-supplemented media, are added to the MLC reaction. This activation process is not observed when PL-generated MSCs are used in the MLC as third party, as shown in greater detail below. We conclude that the MSCs of the invention, cultured in PL-supplemented media are less immunogenic and that growing MSCs in FCS-supplemented media may act as a strong antigen or at least has adjuvant function in T-cell stimulation. This result again is reflected in differential gene expression showing a downregulation of MHC II compounds verifying the decreased immunostimulation by MSC, as shown below.

Moreover, the MSCs of the invention, cultured in PL-supplemented media show upregulation of genes involved in the cell cycle (e.g. cyclins and cyclin dependent kinases) and the DNA replication and purine metabolism when compared to MSCs cultured in FCS-supplemented media. On the other hand, genes functionally active in cell adhesion/extracellular matrix (ECM)-receptor interaction, differentiation/development, TGF-β signaling and TSP-1 induced apoptosis could be shown to be downregulated in the MSCs of the invention, cultured in PL-supplemented media when compared to MSCs cultured in FCS-supplemented media, again supporting the results of faster growth and accelerated expansion.

The MSCs of the invention, cultured in PL-supplemented media when intraaterially administered lead to improvement of regeneration of hypoxic tissue by interfering with the local inflammation, apoptosis and by delivering growth factors needed for the repair of the damaged cells. Hypoxic cells secrete SDF 1 (stromal cell derived factor 1) which attracts MSCs carrying the chemokine receptor 4 (CXCR4). The MSCs of the invention, cultured in PL-supplemented media are particular good candidates for regenerative therapy in CNS damage. They express the gene Prickle1 to an eight-fold higher degree compared to MSCs cultured in FCS-supplemented media which is involved in neuroregeneration. Mouse Prickle1 and Prickle2 are expressed in postmitotic neurons and promote neurite outgrowth (Okuda H, Miyata S, Mori Y, Tohyama M. FEBS Lett. 2007 Oct. 2; 581(24):4754-60). Furthermore, MAG (Myelin-associated glycoprotein) is expressed at 13-fold lower amount in the MSCs of the invention, cultured in PL-supplemented media. MAG is a cell membrane glocoprotein and may be involved in myelination during nerve regereneration. The lack of recovery after central nervous system injury is caused, in part, by myelin inhibitors including MAG. MAG acts as a neurite outgrowth inhibitor for most neurons tested but stimulates neurite outgrowth in immature dorsal root ganglion neurons (Vyas A A, Patel H V, Fromholt S E, Heffer-Lauc M, Vyas K A, Dang J, Schachner M, Schnaar R L. Gangliosides are functional nerve cell ligands for myelin-associated glycoprotein (MAG), an inhibitor of nerve regeneration. Proc Natl Acad Sci USA, 2002; 99(12):8412-7). These differentially regulated genes would favour the use of PL cultured hMSC for regeneration of neuronal injury.

Additionally, the expression of RAR-responsive (TIG1) (retinoid acid (RA) receptor-responsive 1 gene, shows 12 fold higher expression in the MSCs of the invention, cultured in PL-supplemented media) (Liang et al. The quantitative trait gene latexin influences the size of the hematopoietic stromal cell population in mice. Nature Genetics 2007; 39(2):178-188), Keratin 18 (9 fold higher expression in the MSCs of the invention, cultured in PL-supplemented media) (Büler H, Schaller G. Transfection of keratin 18 gene in human breast cancer cells causes induction of adhesion proteins and dramatic regression of malignancy in vitro and in vivo. Mol Cancer Res. 2005; 3(7):365-71), CRBP1 (cellular retinol binding protein 1, 5.7 fold higher expression in the MSCs of the invention, the MSCs of the invention, cultured in PL-supplemented media)(Roberts D, Williams S J, Cvetkovic D, Weinstein J K, Godwin A K, Johnson S W, Hamilton T C. Decreased expression of retinol-binding proteins is associated with malignant transformation of the ovarian surface epithelium. DNA Cell Biol. 2002; 21(1):11-9.) and Prickle 1 suggest a less tumorigenic phenotype of the MSCs of the invention, cultured in PL-supplemented media.

Furthermore, we show evidence, below, that MSCs grown in PL-supplemented medium are more protective against ischemia-reperfusion damage than MSCs grown in FCS-supplemented medium.

Methods of Producing Mesenchymal Stromal Cells

The mesenchymal stromal cells (MSCs) of the invention are cultured in media supplemented with platelet lysate (PL) as opposed to fetal calf serum (FCS). In one embodiment of the method of producing MSCs of the invention, the starting material for the MSCs is bone marrow isolated from healthy donors. Preferably, these donors are mammals. More preferably, these mammals are humans. In one embodiment of the method of producing MSCs of the invention, the bone marrow is cultured on tissue culture flasks between 2 and 10 days prior to washing non-adherent cells from the flask. Optionally, the number of days of culture of bone marrow cells prior to washing non-adherent cells is 2 to 3 days. Preferably the bone marrow is cultured in platelet lysate (PL) containing media. For example, 300 μl of bone marrow is cultured in 15 ml of PL supplemented medium in T75 or other adequate tissue culture dishes.

After washing away the non-adherent cells, the adherent cells are also cultured in media that has been supplemented with platelet lysate (PL). Thrombocytes are a well characterized human product which already is widely used in clinics for patients in need. Thrombocytes are known to produce a wide variety of factors, e.g. PDGF-BB, TGF-b, IGF-1, and VEGF. In one embodiment of the method of producing MSCs of the invention, an optimized preparation of PL is used. This optimized preparation of PL is made up of pooled platelet rich plasmas (PRPs) from at least 10 donors (to equalize for differences in cytokine concentrations) with a minimal concentration of 3×10⁹ thrombocytes/ml.

According to preferred embodiments of the method of producing MSCs of the invention, PL was prepared either from pooled thrombocyte concentrates designed for human use (produced as TK5F from the blood bank at the University Clinic UKE Hamburg-Eppendorf, pooled from 5 donors) or from 7-13 pooled buffy coats after centrifugation with 200×g for 20 min. Preferably, the PRP was aliquoted into small portions, frozen at −80° C., and thawed immediately before use. PL-containing medium was prepared freshly for each cell feeding. In a preferred embodiment, medium contained αMEM as basic medium supplemented with 5 IU Heparin/ml medium (source: Ratiopharm) and 5% of freshly thawed PL. The method of producing MSCs of the invention, uses a method to prepare PL that differs from others according to the thrombocyte concentration and centrifugation forces. The composition of this PL is described in greater detail, below.

In one embodiment of the method of producing MSCs of the invention, the adherent cells are cultured in PL-supplemented media at 37° C. with approximately 5% CO₂ under hypoxic conditions. Preferably, the hypoxic conditions are an atmosphere of 5% O₂. In some situations hypoxic culture conditions allow MSCs to grow more quickly. This allows for a reduction of days needed to grow the cells to 90-95% confluence. Generally, it reduces the growing time by three days. In another embodiment of the method of producing MSCs of the invention, the adherent cells are cultured in PL-supplemented media at 37° C. with approximately 5% CO₂ under normoxic conditions, i.e. wherein the O₂ concentration is the same as atmospheric O₂, approximately 20.9%. Preferably, the adherent cells are cultured between 9 and 12 days, being fed every 4 days with PL-supplemented media. In one embodiment of the method of producing MSCs of the invention, the adherent cells are grown to between 90 and 95% confluence. Preferably, once this level of confluence is reached, the cells are trypsinized to release them from the plate.

In certain embodiments, the population of cells that is isolated from the plate is between 85-95% MSCs. In other embodiments, the MSCs are greater than 95% of the isolated cell population.

In another embodiment of the method of producing MSCs of the invention, the cells are frozen after they are released from the tissue culture plate. Freezing is performed in a step-wise manner in a physiologically acceptable carrier, 20% serum albumin and 10% DMSO. Thawing is also performed in a step-wise manner. Preferably, when thawed, the frozen MSCs of the invention are diluted 4:1 to remove DMSO. If the MSCs are to be administered intra-arterially, the DMSO is diluted from the cells. If the MSCs are to be administered intravenously, then it is not important to dilute the DMSO from the cells. In this case, frozen MSCs of the invention are thawed quickly at 37° C. and administered intravenously without any dilution or washings. Optionally the cells are administered following any protocol that is adequate for the transplantation of hematopoietic stromal cells (HSCs). Preferably, the serum albumin is human serum albumin.

In another embodiment of the method of producing MSCs of the invention, the cells are frozen in aliquots of 10⁶-10⁸ cells in 50 mL of physiologically acceptable carrier and serum albumin (HSA). In another embodiment of the method of producing MSCs of the invention, the cells are frozen in aliquots of 10⁶-10⁸ cells per kg of subject body weight, in 50 mL of physiologically acceptable carrier and serum albumin (HSA). In one aspect of these embodiments, when a therapeutic dose is being assembled, the appropriate number of cryovials is thawed in order to thaw the appropriate number of cells for the therapeutic dose.

Preferably, after DMSO is diluted from the thawed cells, the number of cryovials chosen is placed in a sterile infusion bag with 5% serum albumin. Once in the bag, the MSCs do not aggregate and viability remains greater than 95% even when the MSCs are stored at room temperature for at least 6 hours. This provides ample time to administer the MSCs of the invention to a patient in an operating room. Optionally, the physiologically acceptable carrier is Plasma-lyte. Preferably the serum albumin is human serum albumin. Preferably the albumin is present at a concentration of 5% w/v. Suspending the 10⁶-10⁸ cells MSCs of the invention in greater than 40 mL of physiological carrier is critical to their biological activity. If the cells are suspended in lower volumes, the cells are prone to aggregation. Administration of aggregated MSCs to mammalian subjects has resulted in cardiac infarction. Thus, it is crucial that non-aggregated MSCs be administered according to the methods of the invention. The presence of albumin is also critical because it prevents aggregation of the MSCs and also prevents the cells from sticking to plastic containers the cells pass through when administered to subjects.

In another embodiment of the method of producing MSCs of the invention, a closed system is used for generating and expanding the MSCs of the invention from bone marrow of normal donors. This closed system is a device to expand cells ex vivo in a functionally closed system. In one specific embodiment, the closed system includes: 1. a central expansion unit preferably constructed similarly to bioreactors with compressed (within a small unit), but extended growth surfaces; 2. media bags which can be sterilely connected to the expansion unit (e.g. by welding tubes between the unit and the bags) for cell feeding; and 3. electronic devices to operate automatically the medium exchange, gas supply and temperature.

The advantages of the closed system in comparison to conventional flask tissue culture are the construction of a functionally closed system, i.e. the cell input and media bags are sterile welded to the system. This minimizes the risk of contamination with external pathogens and therefore may be highly suitable for clinical applications. Furthermore, this system can be constructed in a compressed form with consistently smaller cell culture volumes but preserved growth area. The smaller volumes allow the cells to interact more directly with each other which creates a culture environment that is more comparable to the in vivo situation of the bone marrow niche. Also the closed system saves costs for the media and the whole expansion process.

The construction of the closed system may involve two sides: the cells are grown inside of multiple fibres with a small medium volume. In some embodiments, the culture media contains growth factors for growth stimulation, and medium without expensive supplements is passed outside the fibres. The fibres are designed to contain nanopores for a constant removal of potentially growth-inhibiting metabolites while important growth-promoting factors are retained in the growth compartment.

In certain embodiments of the method of producing MSCs of the invention, the closed system is used in conjunction with a medium for expansion of MSCs which does not contain any animal proteins, e.g. fetal calf serum (FCS). FCS has been connected with adverse effects after in vivo application of FCS-expanded cells, e.g. formation of anti-FCS antibodies, anaphylactic or arthus-like immune reactions or arrhythmias after cellular cardioplasty. FCS may introduce unwanted animal xenogeneic antigens, viral, prion and zoonose contaminations into cell preparations making new alternatives necessary.

Methods of Using Mesenchymal Stromal Cells

The MSCs of the invention are used to treat or ameliorate conditions including, but not limited to, stroke, multi-organ failure (MOF), acute renal failure (ARF) of native kidneys, ARF of native kidneys in multi-organ failure, ARF in transplanted kidneys, kidney dysfunction, organ dysfunction and wound repair refer to conditions known to one of skill in the art. Descriptions of these conditions may be found in medical texts, such as The Kidney, by Barry M. Brenner and Floyd C. Rector, Jr., WB Saunders Co., Philadelphia, last edition, 2001, which is incorporated herein in its entirety by reference.

Stroke or cerebral vascular accident (CVA) is a clinical term for a rapidly developing loss of brain function, due to lack of blood supply. The reason for this disturbed perfusion of the brain can be thrombosis, embolism or hemorrhage. Stroke is a medical emergency and the third leading course of death in Western countries. It is predicted that stroke will be the leading course of death by the middle of this century. These factors for stroke include advanced age, previous stroke or ischemic attack, high blood pressure, diabetes, mellitus high cholesterol, cigarette smoking and cardiac arrhythmia with atrial fibrillation. Therefore, a great need exists to provide a treatment for stroke patients.

ARF is defined as an acute deterioration in renal excretory function within hours or days. In severe ARF, the urine output is absent or very low. As a consequence of this abrupt loss in function, azotemia develops, defined as a rise of serum creatinine levels and blood urea nitrogen levels. Serum creatinine and blood urea nitrogen levels are measured. When these levels have increased to approximately 10 fold their normal concentration, this corresponds with the development of uremic manifestations due to the parallel accumulation of uremic toxins in the blood. The accumulation of uremic toxins causes bleeding from the intestines, neurological manifestations most seriously affecting the brain, leading, unless treated, to coma, seizures and death. A normal serum creatinine level is .about.1.0 mg/dL, a normal blood urea nitrogen level is .about.20 mg/dL. In addition, acid (hydrogen ions) and potassium levels rise rapidly and dangerously, resulting in cardiac arrhythmias and possible cardiac standstill and death. If fluid intake continues in the absence of urine output, the patient becomes fluid overloaded, resulting in a congested circulation, pulmonary edema and low blood oxygenation, thereby also threatening the patient's life. One of skill in the art interprets these physical and laboratory abnormalities, and bases the needed therapy on these findings.

MOF is a condition in which kidneys, lungs, liver and heart functions are generally impaired simultaneously or successively, resulting in mortality rates as high as 100% despite the conventional therapies utilized to treat ARF. These patients frequently require intubation and respirator support because their lungs develop Adult Respiratory Distress Syndrome (ARDS), resulting in inadequate oxygen uptake and CO₂ elimination. MOF patients also depend on hemodynamic support, vasopressor drugs, and occasionally, an intra-aortic balloon pump, to maintain adequate blood pressures since these patients are usually in shock and suffer from heart failure. There is no specific therapy for liver failure which results in bleeding and accumulation of toxins that impair mental functions. Patients may need blood transfusions and clotting factors to prevent or stop bleeding. MOF patients will be given stem cell therapy when the physician determines that therapy is needed based on assessment of the patient.

Early graft dysfunction (EGD) or transplant associated-acute renal failure (TA-ARF) is ARF that affects the transplanted kidney in the first few days after implantation. The more severe TA-ARF, the more likely it is that patients will suffer from the same complications as those who have ARF in their native kidneys, as above. The severity of TA-ARF is also a determinant of enhanced graft loss due to rejection(s) in the subsequent years. These are two strong indications for the prompt treatment of TA-ARF with the stem cells of the present invention.

Chronic renal failure (CRF) or Chronic Kidney Disease (CKD) is the progressive loss of nephrons and consequent loss of renal function, resulting in End Stage Renal Disease (ESRD), at which time patient survival depends on dialysis support or kidney transplantation. Need for stem cell therapy of the present invention will be determined on the basis of physical and laboratory abnormalities described above.

In some embodiments of methods of use of MSCs of the invention, the MSCs of the invention are administered to patients in need thereof when one of skill in the art determines that conventional therapy fails. Conventional therapy includes hemodialysis, antibiotics, blood pressure medication, blood transfusions, intravenous nutrition and in some cases, ventilation on a respirator in the ICU. Hemodialysis is used to remove uremic toxins, improve azotemia, correct high acid and potassium levels, and eliminate excess fluid. In other embodiments of methods of use of MSCs of the invention, the MSCs of the invention are administered as a first line therapy. The methods of use of MSCs of the present invention is not limited to treatment once conventional therapy fails and may also be given immediately upon developing an injury or together with conventional therapy.

In certain embodiments, the MSCs of the invention are administered to a subject once. This one dose is sufficient treatment in some embodiments. In other embodiments the MSCs of the invention are administered 2, 3, 4, 5, 6, 7, 8, 9 or 10 times in order to attain a therapeutic effect.

Monitoring patients for a therapeutic effect of the stem cells delivered to a patient in need thereof and assessing further treatment will be accomplished by techniques known to one of skill in the art. For example, renal function will be monitored by determination of blood creatinine and BUN levels, serum electrolytes, measurement of renal blood flow (ultrasonic method), creatinine and inulin clearances and urine output. A positive response to therapy for ARF includes return of excretory kidney function, normalization of urine output, blood chemistries and electrolytes, repair of the organ and survival. For MOF, positive responses also include improvement in blood pressure and improvement in functions of one or all organs.

In other embodiments the MSCs of the invention are used to effectively repopulate dead or dysfunctional kidney cells in subjects that are suffering from chronic renal pathology including chronic renal failure because of the “plasticity” of the MSC populations. The term “plasticity” refers to the phenotypically broad differentiation potential of cells that originate from a defined stem cell population. MSC plasticity can include differentiation of stem cells derived from one organ into cell types of another organ. “Transdifferentiation” refers to the ability of a fully differentiated cell, derived from one germinal cell layer, to differentiate into a cell type that is derived from another germinal cell layer.

It was assumed, until recently, that stem cells gradually lose their pluripotency and thus their differentiation potential during organogensis. It was thought that the differentiation potential of somatic cells was restricted to cell types of the organ from which respective stem cells originate. This differentiation process was thought to be unidirectional and irreversible. However, recent studies have shown that somatic stem cells maintain some of their differentiation potential. For example, hematopoietic stromal cells may be able to transdifferentiate into muscle, neurons, liver, myocardial cells, and kidney. It is possible that as yet undefined signals that originate from injured and not from intact tissue act as trans differentiation signals.

In certain embodiments, a therapeutically effective dose of MSCs is delivered to the patient. An effective dose for treatment will be determined by the body weight of the patient receiving treatment, and may be further modified, for example, based on the severity or phase of the stroke, kidney or other organ dysfunction, for example the severity of ARF, the phase of ARF in which therapy is initiated, and the simultaneous presence or absence of MOF. In some embodiments of the methods of use of the MSCs of the invention, from about 1×10⁵ to about 1×10¹⁰ MSCs per kilogram of recipient body weight are administered in a therapeutic dose. Preferably from about 1×10⁵ to about 1×10⁸ MSCs per kilogram of recipient body weight is administered in a therapeutic dose. More preferably from about 7×10⁵ to about 5×10¹⁰ MSCs per kilogram of recipient body weight is administered in a therapeutic dose. More preferably from about 1×10⁶ to about 1×10⁸ MSCs per kilogram of recipient body weight is administered in a therapeutic dose. More preferably from about 7×10⁵ to about 5×10⁶ MSCs per kilogram of recipient body weight is administered in a therapeutic dose. More preferably about 2×10⁶ MSCs per kilogram of recipient body weight is administered in a therapeutic dose. The number of cells used will depend on the weight and condition of the recipient, the number of or frequency of administrations, and other variables known to those of skill in the art. For example, a therapeutic dose may be one or more administrations of the therapy.

The therapeutic dose of stem cells are administered in a suitable solution for injection. Solutions are those that are biologically and physiologically compatible with the cells and with the recipient, such as buffered saline solution, Plasma-lyte or other suitable excipients, known to one of skill in the art.

In certain embodiments of the MSCs of the invention are administered to a subject at a rate between approximately 0.5 and 1.5 mL of MSCs in physiologically compatible solution per second. Preferably, the MSCs of the invention are administered to a subject at a rate between approximately 0.83 and 1.0 mL per second. More preferably, the MSCs are suspended in approximately 50 mL of physiologically compatible solution and is completely injected into a subject between approximately one and three minutes. More preferably the 50 mL of MSCs in physiologically compatible solution is completely injected in approximately one minute.

In other embodiments, the MSCs are used in trauma or surgical patients scheduled to undergo high risk surgery such as the repair of an aortic aneurysm. Administration of MSCs of the invention to these patients for prophylactic MSC collection and preparation prior to major surgery. In the case of poor outcome, including infected and non-healing wounds, development of MOF post surgery, the patient's own MSCs, prepared according to the methods of the invention, that are cryopreserved may be thawed out and administered as detailed above. Patients with severe ARF affecting a transplanted kidney may either be treated with MSCs, prepared according to the methods of the invention, from the donor of the transplanted kidney (allogeneic) or with cells from the recipient (autologous). Allogeneic or autologous MSCs, prepared according to the methods of the invention, are an immediate treatment option in patients with TA-ARF and for the same reasons as described in patients with ARF of their native kidneys.

In certain embodiments, the MSCs of the invention are administered to the patient by infusion intravenously (large central vein such vena cava) or intra-arterially (via femoral artery into supra-renal aorta). Preferably, the MSCs of the invention are administered via the supra-renal aorta. In certain embodiments, the MSCs of the invention are administered through a catheter that is inserted into the femoral artery at the groin. Preferably, the catheter has the same diameter as a 12-18 gauge needle. More preferably, the catheter has the same diameter as a 15 gauge needle. The diameter is relatively small to minimize damage to the skin and blood vessels of the subject during MSC administration. Preferably, the MSCs of the invention are administered at a pressure that is approximately 50% greater than the pressure of the subject's aorta. More preferably, the MSCs of the invention are administered at a pressure of between about 120 and 160 psi. The shear stressed created by the pressure of administration does not cause injury to the MSCs of the invention. Generally, at least 95% of the MSCs of the invention survive injection into the subject. Moreover, the MSCs are generally suspended in a physiologically acceptable carrier containing about 5% HSA. The HSA, along with the concentration of the cells prevents the MSCs from sticking to the catheter or the syringe, which also insures a high (i.e. greater than 95%) rate of survival of the MSCs when they are administered to a subject. The catheter is advanced into the supra-renal aorta to a point approximately 20 cm above the renal arteries. Preferably, blood is aspirated to verify the intravascular placement and to flush the catheter. More preferably, the position of the catheter is confirmed through a radiographic or sound based method. Preferably the method is transesophageal echocardiography (TEE). The MSCs of the invention are then transferred to a syringe which is connected to the femoral catheter. The MSCs, suspended in the physiologically compatible solution are then injected over approximately one to three minutes into the patient. Preferably, after injection of the MSCs of the invention, the femoral catheter is flushed with normal saline. Optionally, the pulse of the subject found in the feet is monitored, before, during and after administration of the MSCs of the invention. The pulse is monitored to ensure that the MSCs do not clump during administration. Clumping of the MSCs will lead to a decrease or loss of small pulses in the feet of the subject being administered MSCs.

### EXAMPLES

### Example 1

### Preparation of Platelet Lysate

A MSC expansion medium containing platelet lysate (PL) was developed as an alternative to FCS. PL isolated from platelet rich plasma (PRP) were analyzed with either Human 27-plex (from BIO-RAD) or ELISA to show that inflammatory and anti-inflammatory cytokines as well as a variety of mitogenic factors are contained in PL, as shown below in Table 1. The human-plex method presented the concentration in [pg/ml] from undiluted PL while in the ELISA the PL was diluted to a thrombocyte concentration of 1×10⁹/ml and used as 5% in medium (the values therefore have to be multiplied by at least 20). <: below the detection limit. Values with a black background are anti-inflammatory cytokines and cells with a gray background are inflammatory cytokines.

TABLE1
Determination of factor-concentrations in PL.
Human 27-plex (BIO-RAD) [pg/ml]

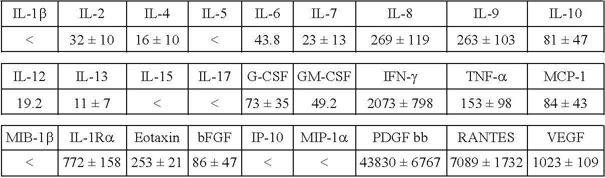

ELISA (n = 6, 5% PL) [pg/ml]

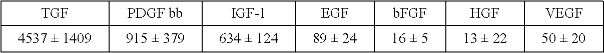

For effective expansion of MSC, an optimized preparation of PL was needed. The protocol includes pooling PRPs from at least 10 donors (to equalize for differences in cytokine concentrations) with a minimal concentration of 3×10⁹ thrombocytes/ml.

PL was prepared either from pooled thrombocyte concentrates designed for human use (produced as TK5F from the blood bank at the University Clinic UKE Hamburg-Eppendorf, pooled from 5 donors) or from 7-13 pooled buffy coats after centrifugation with 200×g for 20 min. The platelet rich plasma (PRP) was aliquoted into small portions, frozen at −80° C., thus obtaining PL and thawed immediately before use. PL-containing medium was prepared fresh for each cell feeding. Medium contained αMEM as basic medium supplemented with 5 IU Heparin/ml medium (source: Ratiopharm) and 5% of freshly thawed PL (Tab. 2).

### Example 2

### Production of Mesenchymal Stromal Cells in Platelet Lysate-Supplemented Media

Bone marrow was collected from non-mobilized healthy donors. White blood cells (WBC) concentrations and CFU-F from bone marrows isolated from different donors varied. This is summarized in Table 3, below.

TABLE 3
Comparison of Different Bone Marrow Donors

| | | | WBC per 50 ml | | |
|---|---|---|---|---|---|
| Donor | Sex | Age | [×10⁸] | Physician | CFU-F/10⁶ cells |
| 1 | M | 60+ | 19.1 | FA | 16 |
| 2 | M | 50+ | 10.1 | AZ | >250 |
| 3 | M | 50+ | 3.1 | AZ | 0.2 |
| 4 | F | | 6.6 | AZ | 50 |
| 5 | M | 37 | 6.4 | Clinical | 60 |
| 6 | M | 29 | 12.1 | NK | 250 |
| 7 | M | | 6.9 | AZ | 62 |
| 8 | F | 40 | 16.8 | FA | 230 |
| 9 | F | 24 | 12.7 | FA | 43 |
| 10 | F | 37 | 11.6 | FA | 225 |
| 11 | M | 24 | 21.1 | FA | 260 |
| 12 | F | 26 | 4.6 | AZ | 47 |
| 13 | F | 25 | 10.1 | FA | 23 |
| 14 | M | | 17.4 | FA | 12 |
| 15 | W | 28 | 11.1 | FA | 130 |

Once the bone marrow was received, a sample was removed and sent for infectious agent testing. Testing will included human immunodeficiency virus, type 1 and 2 (HIV I/II), human T cell lymphotrophic virus, type I and II (HTLV I/II), hepatitis B virus (HBV), hepatitis C virus (HCV), Treponema pallidum (syphilis) and cytomegalovirus (CMV).

Reagents used are shown in Table 4, below.

TABLE 4
Reagents.

| | | | FDA- | | | |
|---|---|---|---|---|---|---|
| Reagent | FinalConcentration | Source | Approved | Vendor | Cat # | COA |
| AlphaMEM | Trace amounts | Non- | Yes | Cambrex | 12-169F | Yes |
| | | mammalian | | | | |
| Platelet Rich | Trace amounts | Human | No | American Red | NA | No |
| Plasma | | | | Cross | | |
| 25% Human | 5% | Human | Yes | NDC 0053- | NA | Yes |
| Serum | | | | 7680-32 | | |
| Albumin | | | | | | |
| Plasmalyte | 40 ml | Non- | Yes | Baxter | 2B2543Q | Yes |
| | | mammalian | | | | |
| Phosphate | Trace amounts | Non- | Yes | SAFC | 59321 | Yes |
| Buffered | | mammalian | | Biosciences | | |
| Saline | | | | | | |
| Trypsin/EDTA | Trace amounts | Non- | Yes | SAFC | 59428C | Yes |
| | | mammalian | | Biosciences | | |
| L-Glutamine | Trace amounts | Non- | No | Cambrex | 16-605 | Yes |
| | | mammalian | | | | |
| Penn/Strep | Trace amounts | Non- | Yes | Stem Cell | 07500 | Yes |
| | | mammalian | | Technologies | | |
| DMSO | Trace amounts | Non- | No | Protide | PP1300 | Yes |
| | | mammalian | | Pharmaceutical | | |

300 μl of whole bone marrow was plated in 15 ml of αMEM media containing 5% PL in tissue culture flask with 75 cm² of growth area or in larger vessels for 6-10 days to allow the mesenchymal stromal cells (MSCs) to adhere. Residual non-adherent cells was washed from the flask. αMEM media containing 5% platelet-rich plasma was added to the flask. Cells were allowed to grow until 70% confluency (approximately 3-4 days). Cells were then trypsinized and re-plated into a Nunc Cell Factory™. Cells remained in the Cell Factory™ for approximately 10-15 days for expansion with media exchanges every 4 days.

Cells were harvested by first washing in phosphate buffered saline (PBS), treating with trypsin and washing with αMEM and then cryopreserved in 10% DMSO, 5% human serum albumin and Plasmalyte using controlled-rate freezing. When the cells were required for infusion, they were thawed, washed free of DMSO and resuspended to the desired concentration in Plasmalyte containing 5% human serum albumin.

The final cell product consisted of approximately 10⁶-10⁸ cells per kg of weight of the subject (depending on the dose schedule) suspended in 50 ml Plasmalyte with 5% Human serum albumin. No growth factors, antibodies, stimulants, or any other substances were added to the product at any time during manufacturing. The final concentration was adjusted to provide the required dose such that the volume of product that is returned to the patient remained constant.

### Example 3

### Comparison of MSCs Grown in Platelet Lysate- and Fetal Calf Serum-Supplemented Media

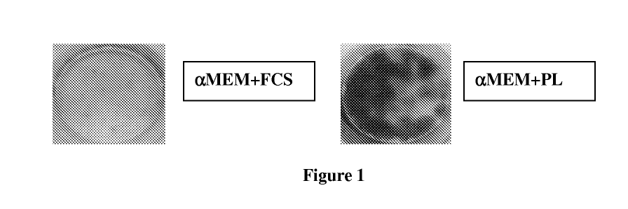

The isolation of MSCs from bone marrow (BM) has been shown to be more effective with PL- compared to FCS-supplemented media. The size (FIG. 1) as well as the number (Table 5) of CFU-F were considerably higher using PL as supplement in the medium (FIG. 1).

TABLE 5
CFU-F from MSCs with FCS- or PL-supplemented media.
Values are shown for 10⁷ plated cells.

| | αMEM + FCS | αMEM + PL |
|---|---|---|
| mean ± SE | 415 ± 97 | 1181 ± 244 |

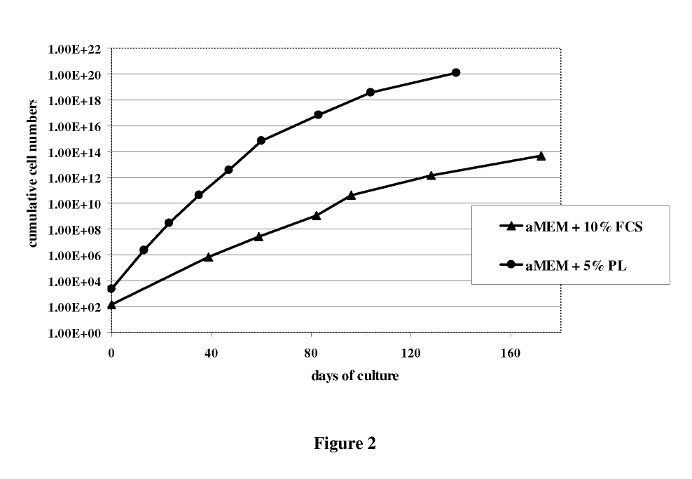

MSCs were isolated by plating 5×10⁵ mononuclear cells/well in 3 ml. FIG. 1 shows are the dark stained CFU-F in FCS- or PL-supplemented media 14 days after seeding. As shown in the graph in FIG. 2, the more effective isolation of MSCs with PL-supplemented media is followed by a more rapid expansion of these cells over the whole cultivation period until senescence.

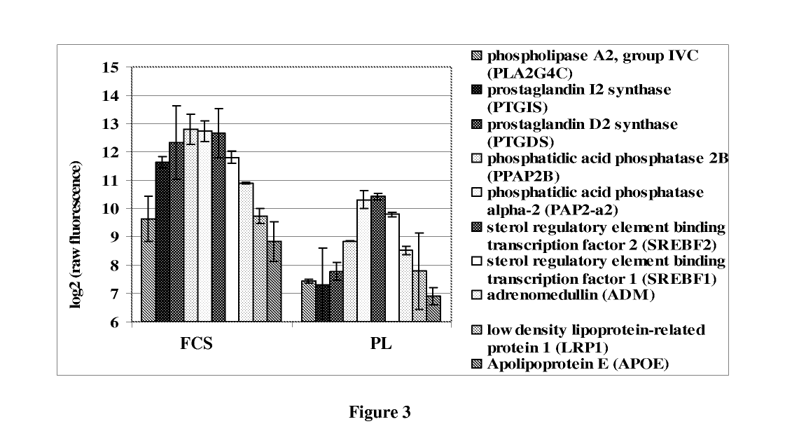

Also, MSCs cultured in PL-supplemented media are less adipogenic in character when compared to MSCs cultured in FCS-supplemented media. FIG. 3 shows the downregulation of genes involved in fatty acid metabolism in MSCs cultured in PL-supplemented media compared to MSCs cultured in FCS-supplemented media.

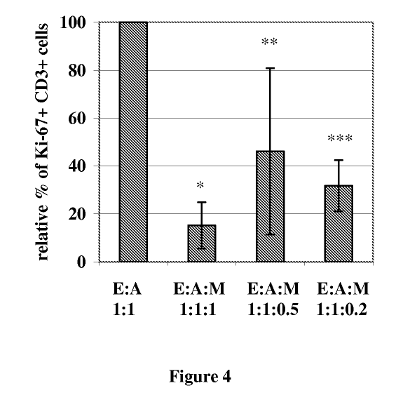

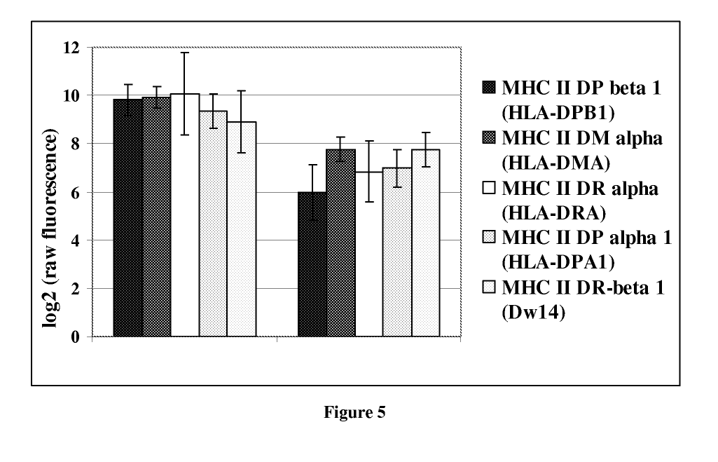

MSC have been described to act immunomodulatory by impairing T-cell activation without inducing anergy. A dilution of this effect has been shown in vitro in mixed lymphocyte cultures (MLC) leading eventually to an activation of T-cells if decreasing amounts of MSC are added to the MLC reaction. This activation process is not observed when PL-generated MSC are used in the MLC as third party. FIG. 4, shows that MSCs cultured in PL-supplemented media are not immunodulatory in vitro even at low numbers (p-values: (*) 4×10⁻⁶; (**) 0.013; (***) 1.9×10⁻⁵; E: effector; A: irradiated activator; M: MSC). Thus, MSCs are less immunogenic after PL-expansion and FCS seems to act as a strong antigen or at least has adjuvant function in T-cell stimulation. This result is also reflected in differential gene expression showing a downregulation of MHC II compounds verifying the decreased immunostimulation by MSC as shown in FIG. 5.

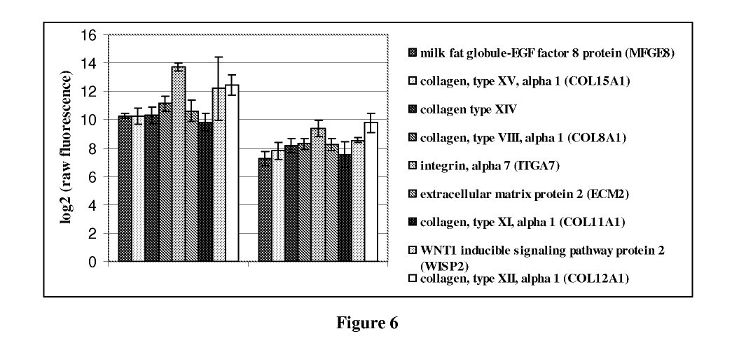

Additional data from differential gene expression analysis of PL-generated compared to FCS-generated MSC showed an upregulation of genes involved in the cell cycle (e.g. cyclins and cyclin dependent kinases) and the DNA replication and purine metabolism. On the other hand, genes functionally active in cell adhesion/extracellular matrix (ECM)-receptor interaction (FIG. 6), differentiation/development, TGF-β signaling and thrombospondin induced apoptosis could be shown to be downregulated in PL-generated MSC, again supporting the results of faster growth and accelerated expansion.

Furthermore, we show evidence that MSCs grown in PL-supplemented medium are more protective against ischemia-reperfusion damage than MSCs grown in FCS-supplemented medium. Human kidney proximal tubular cells (HK-2) were forced to start apoptotic events by incubation with antimycin A, 2-deoxyclucose and calcium ionophore A23187 (Lee H T, Emala C W 2002, J Am Soc Nephrol 13, 2753-2761; Xie J, Guo Q 2006, J Am Soc Nephrol 17, 3336-3346). This treatment chemically mimics an ischemic event. Reperfusion was simulated by refeeding the HK-2 cells with rescue media consisting of conditioned medium incubated for 24 h on confluent layers of MSCs grown with either alphaMEM+10% FCS or alphaMEM+5% PL.

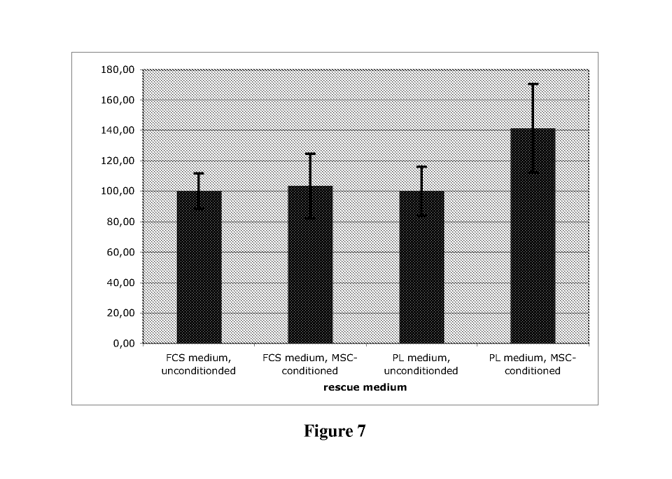

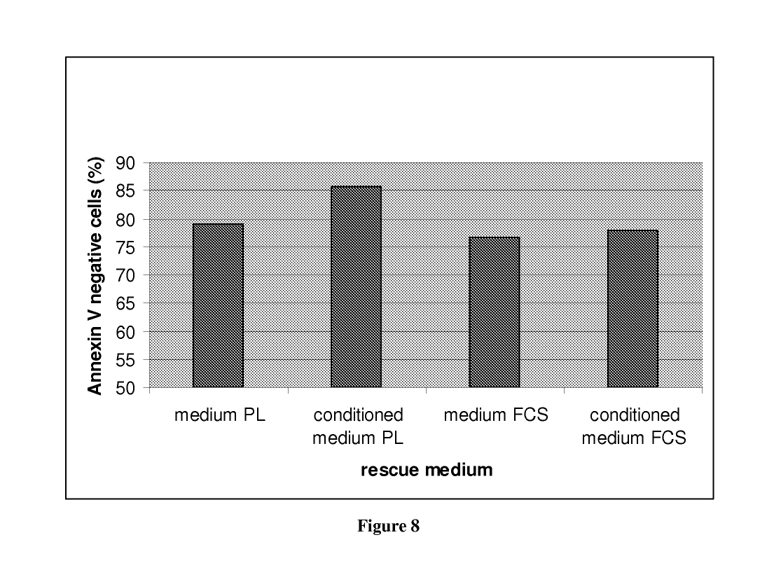

The obtained results show that supernatants from MSCs grown in PL-containing medium are more effective to reduce HK-2 cell death after chemically simulated ischemia/reperfusion than supernatants from MSCs grown in FCS-supplemented medium (FIG. 7).

A parallel FACS assay detecting annexin V which binds to apoptotic cells showed similar results. The proportion of viable cells (=annexin V negative) was highest in the HK-2 cells rescued with MSC-conditioned PL medium (85.7%, as compared to 78.0% in MSC-conditioned FCS medium, FIG. 8). Thus, it appears that PL-MSCs contain a higher rate of factors that prevent kidney tubular cells from dying after ischemic events and/or less factors that promote cell death compared to FCS-MSC conditioned medium. Thus, PL appears to be the supplement of choice to expand MSCs for the clinical treatment of ischemic injuries.

### Example 4

### Cryospreservation Protocol for Human Mesenchymal Stromal Cells (hMSCs)

Mesenchymal stromal cells were cryopreserved in a DMSO solution, at a final concentration of 10%, for long-term storage in vapor phase liquid nitrogen (LN2, <−150° C.). The viability and functionality of hMSCs in prolonged storage has been demonstrated and there is currently no recognized expiration of products that remain in continuous LN2 storage.

hMSCs were derived from human bone marrow.

Reagents, Standards, Media, and Special Supplies Required:

| | |
|---|---|
| Dimethyl Sulfoxide (DMSO) | Protide Pharmaceuticals |
| Human Serum Albumin 25% | NDC 0053-7680-32 |
| Plasmalyte A | |
| Cryovials | |
| Dispensing Pin | |
| 20 cc Syringe without Needle | |
| 30 cc Syringe without Needle | |
| 18 gauge Blunt Fill Needle | |
| Alcohol Preps | |
| Betadine Preps | |
| Ice Bucket | |
| 10 ml serological pipette | |
| 25 ml serological pipette | |
| 250 ml Conical Tube | |
| Cryogloves | |

Instrumentation:

### Pipettes

### Biological Safety Cabinet (BSC)

### Controlled Rate Freezer (CRF)

LN2 Storage Freezer with Inventory System

### Centrifuge

A. Calculate the Number of Cyrovials Needed to Freeze the hMSC Product

1. Calculating Freeze Mix: The number of cryovials necessary to freeze a give quantity of cells was calculated. The cells are stored at 15×10⁶/ml. Thus, the number of cells present was divided by this number to ascertain the volume of cells and medium to be frozen.

For example, 3.71×10⁸=24.7 ml.

2. Calculating number of cryovials: The number of vials needed for a given volume of cells plus medium was calculated. The volume of the cryovials was 1 ml or 4 ml. Thus, the volume calculated above was divided into the number of cryovials needed.

For example: 24 ml=6, 4 ml cyrovials

B. Calculate the Total Freeze Volume

Total freeze volume consisted of 10% DMSO by volume, 20% albumin by volume, and the remaining volume Plasmalyte (70%).
For example: Total Freeze Volume=24 ml
   DMSO=2.4 ml
   Albumin=4.8 ml
   Plasmalyte=16.8 ml

C. Prepare Freeze Mix

1. Ice bucket prepared.
2. The desired volume of DMSO was obtained with an appropriate sized syringe.
3. The same volume of plasmalyte that was obtained.

a. e.g. 6 ml of DMSO, 6 ml of plasmalyte

4. The DMSO and plasmalyte were added to the “Freeze Mix” tube.
5. The solution was mixed and placed on ice to chill for at least 10 minutes.
6. The albumin was placed on ice

D. Prepare Sample for Freezing

1. The final product was centrifuged in a 250 ml conical tube at 600×g (˜1600 rpm) for 5 minutes, no brake.
2. The supernatant was removed to one inch above the cell pellet using a 25 ml serological pipette, The cell pellet was not disturbed.
3. The supernatant was removed and placed in a sterile 250 ml conical tube labeled “Sup”.
4. Both the cells and supernatant were placed on ice

E. Freezing

1. The amount of plasmalyte still needed for the freeze mix was calculated and the desired volume was obtained.

a. For example, the volume of DMSO+the volume of already added plasmalyte+the volume of albumin+cell pellet volume minus the total freeze volume equals amount of plasmalyte needed.

2. The albumin bag was aseptically spiked with a dispensing pin and the desired volume of albumin was removed.
3. The albumin and plasmalyte were added to the “Freeze Mix” tube and mixed.
4. Using a 10 ml serological pipette the chilled freeze mix aseptically removed and added slowly to the resuspended cells. While adding the freeze mix cells were gently mixed by swirling. Once the Freeze Mix was added to the product, the freeze was initiated within 15 minutes. If a delay was expected, the product mixture was placed back on ice. Under no circumstances was the mix allowed to be unfrozen for more than 30 minutes.
5. The lid was placed on the tube containing cell mix and the tube was inverted several times to mix the contents.
6. Using a 10 ml serological pipette the freeze volume was aseptically removed and the appropriate volume was dispensed into each labeled cryovial. In 1.8 ml vials 1 ml of cell mix was placed. In 4.5 ml vials 4 ml of cell mix was placed.
7. The cryovials were then immediately placed on ice and then frozen using the controlled rate freezer to −80° C.

F. Expected Ranges for MSCs Thawed after being Frozen According to Protocol:

### 1. Thawed Product Viability≧70%

### 2. Sterility Testing=Negative

3. Differentiation=growth for adipogenic, osteogenic, and chondrogenic
4. Flow cytometry

a. CD105 (≧95%)

b. CD 73 (≧95%)

c. CD 90 (≧95%)

d. CD 34 (<2%)

e. CD 45 (<2%)

f. HLA-DR (<2%)

### 5. Endotoxin<5.0 EU/kg

6. Mycoplasma=negative

### Example 5

### Thawing Protocol for Human Mesenchymal Stromal Cells (hMSCs)

Stored human Mesenchymal stromal cells (hMSC) are cryopreserved using DMSO as a cell cryoprotectant. When thawed, DMSO creates a hypertonic environment which leads to sudden fluid shifts and cell death. To limit this effect, the product was washed with a hypertonic solution ameliorating DMSO's unfavorable effects. Post-thaw product release testing was done to ensure processing was performed so as to prevent contamination or cross-contamination.

Reagents, Standards, Media, and Special Supplies Required:

| | |
|---|---|
| Human Serum Albumin (HSA) 25% | NDC 52769-451-05 |
| Plasmalyte A | |
| Trypan Blue | |
| 300 ml Transfer Pack | |
| 15 ml conical tube | |
| 50 ml conical tube | |
| 250 ml Conical Tube | |
| 150 ml Transfer Pack | |
| Sterile Transfer Pipette | |
| 1.5 Eppendorf tube | |

Red Top Vacutainer Tubes or equivalent
10 cc syringe
20 cc syringe
30 cc syringe
60 cc syringe
5 ml serological pipette
10 ml serological pipette

### Ice Bucket

### Blunt End Needle

200-1000 μl sterile tips

### Cryogloves

### Biohazard Bag

### Iodine

Alcohol wipes

Instrumentation:

### Biological Safety Cabinet (BSC)

### Centrifuge

### Sterile Connecting Device

### Microscope, Light

### Thermometer

### Water Bath

### Hemacytometer

### Pipettes

Computer with Freezerworks

### Ambient Shipper

A. Wash Solution Preparation

1. The cell dose required for infusion was calculated based on the recipient's weight. The required number of cells for infusion based on recipient weight was calculated by multiplying the cell dosage per kg times the recipient weight in kg to arrive at the number of cells necessary.
2. The number of cryovials needed to achieve the calculated cell dose was then determined a. 1 ml of cell mix contains 15×10⁶ cells.
3. The wash solution volume needed to thaw all required cryovials was then calculated: For the example below, all numbers listed below are for a 100 kg patient.

a. Volume of product, multiplied times 4 in addition to 80 mls for cell resuspension and testing
1) for a dose of 7×10⁵ cells=˜7 mls of product thawed and a wash solution volume of 108 ml was used;
2) for a dose of 2×10⁶ cells=˜19 mls of product thawed and a wash solution volume of 156 ml was used;
3) for a dose of 5×10⁶ cells=˜46 mls of product thawed and a wash solution volume of 264 ml was used.

b. Wash Solution=20% by volume stock albumin (25% Human, USP, 12.5 g/50 ml), 80% Plasmalyte

4. A female end was sterile connected to a 300 ml transfer pack.
5. Using sterile technique, a calculated volume of Plasmalyte was removed and placed in a transfer pack.
6. The calculated volume of albumin was removed and the volume added to the Plasmalyte.
7. The bag was mixed well, placed in a tube on ice and solution was allowed to chill for at least 10 minutes

B. Thawing and Washing

1. The exterior of the cryovial containing the hMSCs was wiped with 70% alcohol and placed in a bucket with ice.
2. Each vial was thawed one at a time
3. The vial was wiped down with 70% alcohol and place in the biological safety cabinet.
4. Using a 5 ml serological pipette thawed product was removed and place in the labeled “Thaw and Washed Product” tube.
5. Using an appropriate sized serological pipette the required amount of wash solution was removed (vial volume times 4).

a. The wash solution was slowly added drop wise to the thawed product. The was solution was gradually introduced to the cells while gently rinsing the product to allow the cells to adjust to normal osmotic conditions. Slow addition of wash solution with gentle agitation prevents cell membrane rupture from osmotic shock during thaw.

b. 1 ml of the wash solution was used to rinse the cryovial.

c. The rinse was added to the product conical tube.

6. The conical tube was placed on ice and retrieve the next vial
7. Steps 1-5 were repeated for any remaining vials.

a. For higher doses the volume was split in half, with one half of the volume thawed in one 250 ml conical tube and the other half in the other 250 ml conical tube.

8. The Thaw and Washed Product tube was centrifuged at 500 g for 5 min. with the brake on slow.
9. A serological pipette was used to slowly remove the supernatant (approximately one inch from the cell pellet)
10. The cell pellet was resuspended in 5 ml of wash solution.

a. For higher doses
1) The cell pellets were resuspended in the remaining supernatant
2) The cell pellets were combined.
3) 5 ml of wash solution was used to rinse the conical tube in which the cell pellet was removed and add wash solution to the product.

1. A population of mesenchymal stromal cells produced by
(a) providing bone marrow;
(b) culturing the bone marrow on tissue culture plates in culture media between 2 and 10 days;
(c) removing non-adherent cells;
(d) culturing the adherent cells between 9 and 20 days in platelet lysate supplemented media; and
(e) removing the adherent cells from the tissue culture plates;
thereby producing a population of mesenchymal stromal cells.

2. The population of claim 1, wherein the population of mesenchymal stromal cells are a mammalian population of mesenchymal stromal cells.

3. The population of claim 2, wherein the mammalian population of mesenchymal stromal cells is a human population of mesenchymal stromal cells.

4. The population of claim 1, wherein the platelet lysate is present in the culture media at about 20 μl of platelet lysate per 1 ml of culture media.

5. The population of claim 1, wherein the platelet lysate is made up of pooled thrombocyte concentrates or pooled buffy coats after centrifugation.

6. A population of mesenchymal stromal cells wherein the population mesenchymal stem cells have been cultured in platelet lysate supplemented culture media and wherein the population of mesenchymal stromal cells expresses Pickle1 at a higher degree than mesenchymal stromal cells that have been cultured in fetal calf serum supplemented culture media.

7. The population of claim 6, wherein the population mesenchymal stem cells that have been cultured in platelet lysate are less immunogenic than mesenchymal stromal cells that have been cultured in fetal calf serum supplemented culture media.

8. A population of mesenchymal stromal cells wherein the population expresses the antigens CD105, CD90, CD73 and MHC I on its surface.

9. The population of claim 8, wherein the population does not express a protein selected from the group consisting of CD45, CD34 and CD14 on its surface.

10. A method of treating a neurological, inflammatory or renal disorder in a subject in need by administering a therapeutically effective dose of a population of mesenchymal stromal cells isolated by the method comprising:
(a) providing bone marrow;
(b) culturing the bone marrow on tissue culture plates in culture media between 2 and 10 days;
(c) removing non-adherent cells;
(d) culturing the adherent cells between 9 and 20 days in platelet lysate supplemented media; and
(e) removing the adherent cells from the tissue culture plates;
thereby treating a neurological, inflammatory or renal disorder.

11. The method of claim 10, wherein the disorder is a neurological disorder.

12. The method of claim 11, wherein the neurological disorder is stroke.

13. The method of claim 10, wherein the disorder is an inflammatory disorder.

14. The method of claim 13, wherein the inflammatory disorder is multi-organ failure.

15. The method of claim 10, wherein the disorder is a renal disorder.

16. The method of claim 15, wherein the renal disorder is selected from the group consisting of acute renal failure, chronic renal failure and chronic kidney disease.

17. A method of isolating a population of mesenchymal stromal cells comprising
(a) providing bone marrow;
(b) culturing the bone marrow on tissue culture plates in culture media between 2 and 10 days;
(c) removing non-adherent cells;
(d) culturing the adherent cells between 9 and 20 days in platelet lysate supplemented media; and
(e) removing the adherent cells from the tissue culture plates;
thereby isolating a population of mesenchymal stromal cells.

18. The method of claim 17, wherein the population of mesenchymal stromal cells are a mammalian population of mesenchymal stromal cells.

19. The method of claim 18, wherein the mammalian population of mesenchymal stromal cells is a human population of mesenchymal stromal cells.

20. The method of claim 17, wherein the platelet lysate is present in the culture media at about 20 μl of platelet lysate per 1 ml of culture media.

21. The population of claim 17, wherein the platelet lysate is made up of pooled thrombocyte concentrates or pooled buffy coats after centrifugation.
